(19) United States
(12) Patent Application Publication
(10) Pub. No.: US 2009/0191160 A1
(43) Pub. Date: Jul. 30, 2009

(54) Methods of producing pluripotent stem-like cells

(75) Inventor: Yiling Hong, Centerville, OH (US)

Correspondence Address: EDWARDS ANGELL PALMER & DODGE LLP, P.O. BOX 55874, BOSTON, MA, 02205, US

(73) Assignee: University of Dayton

(21) Appl. No.: 12/228,205

(22) Filed: Aug. 11, 2008

Related U.S. Application Data
(60) Provisional application No. 60/955,169, filed on Aug. 10, 2007.
(60) Provisional application No. 60/994,212, filed on Sep. 18, 2007.

(51) Int. Cl. A61K 38/18; C12N 5/02; A61K 35/12; A61P 35/00; C12N 5/10

(52) U.S. Cl. 424/93.7; 435/378; 435/325; 514/12

(57) ABSTRACT

The instant invention provides methods and compositions for the production and use of pluripotent stem-like cells from somatic cells, e.g., fibroblasts.

### RELATED APPLICATIONS

This application claims the benefit of U.S. Provisional Application No. 60/994,212, filed Sep. 18, 2007 and U.S. Provisional Application No. 60/955,169, filed Aug. 10, 2007. The entire contents of each of the aforementioned applications is hereby incorporated by reference.

### BACKGROUND

Stem cells are cells having the ability to divide to an unlimited extent and to differentiate under suitable circumstances and/or through suitable stimuli to form different types of cells. Stem-cells have the potential to develop into cells with a characteristic shape and specialized functions.

The use of human embryos to derive stem-like cells has raised significant ethical concerns and has promoted the search for methods of producing pluripotent cells from somatic cells. It has been demonstrated that fully differentiated cells can reverse their gene expression profile to that of pluripotent cells (Alberio et al. Reproduction 132:709-720). Adult somatic cells can be reprogrammed after fusion with a mature oocyte, and such reprogrammed cells have been used to produce cloned animals of different species (Wilmut et al. Nature 385:810-813; Wakayama et al. Nature 394:369-374). The successes of such processes provide evidence that somatic nuclei can be reprogrammed to a pluripotent state by the factors in the oocyte cytoplasm, and the reprogrammed nuclei can direct embryonic development to term. Recent reports showed that the reprogramming of mouse fibroblasts to a pluripotent state can be achieved in vitro by ectopic expression of four transcription factors, Oct4, Sox2, c-Myc and Klf4, and these induced pluripotent stem-like cells are indistinguishable from embryonic stem (ES) cells (Takahashi et al. Cell 126:663-676; Okita et al. Nature 148:313-317; Wernig et al. Nature 448:318-324). Several other reprogramming strategies have also been reported, such as ES cell and somatic cell fusion (Tada et al. Developmental Dynamics 227:504-510), and injection of ES cell extracts into somatic cells (Taranger et al. Molecular and Cellular Biology 16:5719-55735).

The use of serum replacement (SR)-containing medium has previously been reported as effective for growth and maintenance of undifferentiated stem cells under serum-free conditions (Lansdown. Curr. Probl Dermatol. 33:17; Webster et al. Clinical Orthopedics and Related Research 161:105). It has also been shown that ES cells grown in SR-containing media are less differentiated than those grown in serum-containing medium, and that the medium can both improve the efficiency of establishing stem cell lines from blastocysts and increase the success rate of producing chimaeric mice (Lansdown et al. Br. J Dermatol. 137:728; Becker. Neuro Rehabilitation, 17:23-31). However, to date, there has been little description of a role for SR medium in cellular reprogramming.

In view of the ethical objections regarding embryonic stem-cells, there is a need to develop alternative methods for creating or isolating stem-like cells for research and therapeutic use.

### SUMMARY OF THE INVENTION

The instant invention provides, at least in part, methods for producing stem-like cells, e.g., pluripotent stem-like cells, from somatic cells, e.g., fibroblasts, through induction by arachadonic acid and a serum albumin. In certain embodiments, these factors are present in serum replacement media.

Accordingly, in one aspect, the instant invention provides methods of producing a stem-like cell by ulturing a somatic cell in the presence of a lipid, e.g., arachadonic acid, and serum albumin (SA); thereby producing a stem-like cell. In a related embodiment, the method further comprises isolating a stem-like cell, e.g., isolating the stem-like cell based on the expression of one or more markers.

In another embodiment, the stem-like cell is a pluripotent stem-like cell. In a related embodiment, the serum albumin is bovine serum albumin (BSA). In one exemplary embodiment, the BSA is high lipid BSA. In another embodiment, the arachodonic acid BSA are present in a serum replacement (SR) medium.

In another embodiment, the somatic cell is a fibroblast, e.g., a human or mouse fibroblast.

Exemplary fibroblasts are human dermal skin fibroblasts, mouse embryonic skin fibroblasts, or mouse adult skin fibroblast.

In another embodiment, the methods further comprise contacting the cultured cells with a protease, e.g., trypsin.

In another aspect, the instant invention provides methods for producing pluripotent stem-like cells by culturing a somatic cell in the presence of arachadonic acid and serum albumin (SA), thereby producing a pluripotent stem-like cell.

In a related embodiment, the method further comprises isolating a stem-like cell.

In another embodiment, the stem-like cell is a pluripotent stem-like cell. In a related embodiment, the serum albumin is bovine serum albumin (BSA). In one exemplary embodiment, the BSA is high lipid BSA. In another embodiment, the arachodonic acid BSA are present in a serum replacement (SR) medium.

In another embodiment, the somatic cell is a fibroblast, e.g., a human or mouse fibroblast.

Exemplary fibroblasts are human dermal skin fibroblasts, mouse embryonic skin fibroblasts, or mouse adult skin fibroblast.

In another embodiment, the methods further comprise contacting the cultured cells with a protease, e.g., trypsin. In another embodiment, the methods of the invention further comprise culturing the somatic cells in the presence of bFGF.

In yet other embodiments of the invention, the methods further comprise culturing the somatic cells in the presence of metal ions. In an exemplary embodiment, the methods comprise the use of silver ions from silver salts such as AgNO₃.

In another aspect, the instant invention provides stem-like cells produced by the method described herein. In one embodiment, the stem-like cells are pluripotent stem-like cells. In one embodiment, the pluripotent stem-like cells are derived from somatic cells, e.g., fibroblasts.

In another aspect, the instant invention provides methods of treating a subjects by contacting the stem-like cells described herein with a tissue-specific growth factor; and administering the cells to the subject.

In another aspect, the instant invention provides methods of treating a subjects by administering to the subject a stem cell derived by the methods described herein, thereby treating the subject. In one embodiment, the subject has a cell proliferative disorder, e.g., cancer.

In another embodiment, the methods further comprise contacting the stem cell with an agent that induces differentiation of the cell into a desired cell type.

In yet another aspect, the invention provides kits comprising an agent to dedifferentiate a somatic cell into a pluripotent stem-like cell and instructions for use.

### BRIEF DESCRIPTION OF THE FIGURES

FIGS. 1a-1e demonstrate that SR-containing medium induced the reprogramming of mouse embryonic skin fibroblast cells into stem cell-like cells. FIG. 1a shows phase contrast images demonstrating the appearance of bright-edged granulated cells 24 hours after commencement of fibroblast induction by SR-containing medium. FIG. 1b shows attachment to the plate of stem-cell like colonies 48 hours after commencement of induction. FIG. 1c shows P5 colonies cultured in SR-containing medium with a feeder layer. FIG. 1d shows P5 colonies cultured in serum-containing medium with a feeder layer. FIG. 1e shows the karyotypes of skin fibroblast cells and ES-like cells. Images were acquired by inverted microscope (Nikon TS100) at 10× magnification using MetaMorph Imaging Software. Scale bar is 100 μm.

FIG. 2 shows that lipid-rich BSA (e.g., AlbuMax I, Invitrogen) in SR-containing medium promoted the dedifferentiation of skin fibroblast cells into stem cell-like cells. The left panel shows results of treatment with basic medium only; the center panel displays results for basic medium with SR; and the right panel shows results for treatment with basic medium containing lipid-rich BSA. Images were acquired by inverted microscope (Nikon TS100) at 10× magnification using MetaMorph Imaging Software. Scale bar is 100 μm.

FIGS. 3a-3d demonstrate that lipid-rich BSA induced Ca²⁺ influx, fibroblast growth factor receptor 3 (FGFR3) expression and demethylation of the Oct4 promoter. FIG. 3a shows that the Ca²⁺ ATPase inhibitor Thapsigargin prevented conversion of the fibroblast cells into stem-like cells. FIG. 3b shows Western blotting for FGF Receptor-3 (FGFR3) protein, revealing expression of FGFR3 following transfer of fibroblast cells into serum-free medium. Lanes 1-4 show results at timepoints of 2 hours, 4 hours, 8 hours and 12 hours after media culture, respectively. β-actin was used as a loading control. FIG. 3c shows that Thapsigargin (1 ug/ml) down-regulated FGF receptor 3 (FGFR3) expression, with lanes 1 and 2 showing the 24 hour timepoint following culture in media, in either the absence or presence of Thapsigargin (1 ug/ml), respectively. β-actin was used as a loading control. FIG. 3d shows phase contrast images of skin fibroblast cells cultured under different conditions, with the left-hand panel showing results for serum-free stem cell medium, the center panel showing the result for serum-free medium culture with 1.5 ul of DMSO, and the right-hand panel showing the result for serum-free medium culture with Thapsigargin (1 ug/ml), where 1000× Thapsigargin stock was dissolved in DMSO. All Images were acquired in the inverted microscope (Nikon TS100) at 10× magnification using the MetaMorph Imaging Software, and the scale bar is 100 μm.

FIGS. 4a,4b and 4c show expression of stem cell markers in SiES cells. FIG. 4a shows Western blot results for the expression of stem cell marker proteins Oct4, Nanog, and Sox2, with iPS cells compared to fibroblast cells. In performing the experiments, cells were lysed with RIPA buffer, and equal amounts of cell lysate were analyzed by Western blot with antibodies. Lane 1 shows fibroblast cell lysate; lanes 2-4 show iPS cell lysate 2 hours, 4 hours and 8 hours after transfer to SR containing medium, respectively. β-actin was used as a loading control.

FIG. 4b shows the results of Alkaline-phosphatase and immunofluorescence staining with SSEA3 and SSEA4 antibodies, which showed that these iES cells expressed important stem cell markers. The AP staining image was captured by microscope (Olympus CK2) via QCapture Pro Imaging Software using 20× magnification, while SSEA3 and SSEA4 immunofluorescence staining images were acquired by inverted microscope (Nikon TS100) at 20× magnification using MetaMorph Imaging Software. The Illumina Microarray global gene expression analysis of the different passages of SiES cells P5 and P15 compared with mouse embryonic stem cells and MEF cells. The heat map of top 10,000 differentially expressioned genes indicated that the SiES at their passage 5 was less similarity to a mES gene expression, while the at their passage 15, the SR-iPS is very similar to embryonic stem cells ES cells.

FIG. 5 Demonstrates developmental potential in vitro and in vivo. FIG. 5a, In vitro differentiation of the embryonic bodies into three germ layers induced by retinonic acid. Differentiated cells were stained with differentiation markers: endoderm a-fetoprotein (AFP), mesoderm smooth muscle (SM-Actin) (middle), and ectoderm (β-tubulin III). FIG. 5b showed cardiomyocyte and smooth muscle derived from SR-iPS cells induced by growth factors. The cells were stained with troponin C and smooth muscle actin. Images were acquired with a Fluoview laser scanning confocal microscope (20× magnification). Scale bar 100 μm. FIG. 5c. A 3-week chimaeric mice produced by injecting the P12 SR-iPS into the black C57 blastcyst.

### DETAILED DESCRIPTION

The instant invention is based, at least in part, on the discovery by the instant inventor that arachadonic acid and serum albumin can be used to reprogram somatic cells, e.g., fibroblasts, to pluripotent stem-like cells that are essentially identical to embryonic stem (ES) cells. Thus, in one aspect, the instant invention provides a method for de-differentiation of fibroblast cells into stem-like cells by contacting the fibroblast cells with arachadonic acid and serum albumin.

In another aspect, the instant invention is based, at least in part, on the discovery by the inventor that agents that stimulate calcium release can be used to dedifferentiate somatic cells, e.g., fibroblasts, into stem-like cells, e.g., pluripotent stem-like cells. In yet another aspect, the instant invention is based, at least in part, on the discovery by the instant inventor that agents that cause an increase in Ca²⁺ ATPase activity can be used to dedifferentiate somatic cells, e.g., fibroblasts, into stem-like cells, e.g., pluripotent stem-like cells.

In another aspect, the methods of the invention comprise contacting a somatic cell, e.g., a fibroblast, with an agent that increase the expression of fibroblast growth factor receptors (FGFRs), thereby creating a pluripotent stem cell.

Before further defining the invention, the following terms are defined for convenience.

The terms “pluripotent stem cell”, “pluripotent stem-like cells”, “stem-like cells” and “stem cells”, and any variants not specifically listed, may be used herein interchangeably, and as used throughout the present application and claims extends to those cell(s) and/or cultures, clones, or populations of such cell(s) which are derived from somatic cells, e.g., fibroblasts, are capable of self regeneration and capable of differentiation to cells of endodermal, ectodermal and mesodermal lineages. As used herein, “pluripotent” refers to cells that can give rise to any cell type except the cells of the placenta or other supporting cells of the uterus. The stem-like cells of the invention may have one or more properties of stem-like cells with out having all properties.

The pluripotent stem-like cell(s) of the present invention are lineage uncommitted, i.e., they are not committed to any particular germ layer, e.g., endoderm, mesoderm, ectoderm, or notochord. They can remain undifferentiated. They can also be stimulated by particular growth factors to proliferate. If activated to proliferate, pluripotent stem-like cells are capable of extended self-renewal as long as they remain lineage-uncommitted.

“Lineage-commitment” refers to the process by which individual cells commit to subsequent and particular stages of differentiation during the developmental sequence leading to the formation of an organism. Lineage commitment can also be induced in vitro, and in such cases it will not lead to the formation of an organism.

The term “lineage-uncommitted” refers to a characteristic of cell(s) whereby the particular cell(s) are not committed to any next subsequent stage of differentiation (e.g., germ layer lineage or cell type) of the developmental sequence.

The term “lineage-committed” refers to a characteristic of cell(s) whereby the particular cell(s) are committed to a particular next subsequent stage of differentiation (e.g., germ layer lineage or cell type) of the developmental sequence. Lineage-committed cells, for instance, can include those cells which can give rise to progeny limited to a single lineage within a germ layers, e.g., liver, thyroid (endoderm), muscle, bone (mesoderm), neuronal, melanocyte, epidermal (ectoderm), etc.

“Pluripotent endodermal stem cell(s)” are capable of self renewal or differentiation into any particular lineage within the endodermal germ layer. Pluripotent endodermal stem-like cells have the ability to commit within endodermal lineage from a single cell any time during their life-span. This commitment process necessitates the use of general or specific endodermal lineage-commitment agents. Pluripotent endodermal stem-like cells may form any cell type within the endodermal lineage, including, but not limited to, the epithelial lining, epithelial derivatives, and/or parenchyma of the trachea, bronchi, lungs, gastrointestinal tract, liver, pancreas, urinary bladder, pharynx, thyroid, thymus, parathyroid glands, tympanic cavity, pharyngotympanic tube, tonsils, etc.

“Pluripotent mesenchymal stem cell(s)” are capable of self renewal or differentiation into any particular lineage within the mesodermal germ layer. Pluripotent mesenchymal stem-like cells have the ability to commit within the mesodermal lineage from a single cell any time during their life-span. This commitment process necessitates the use of general or specific mesodermal lineage-commitment agents. pluripotent mesenchymal stem-like cells may form any cell type within the mesodermal lineage, including, but not limited to, skeletal muscle, smooth muscle, cardiac muscle, white fat, brown fat, connective tissue septae, loose areolar connective tissue, fibrous organ capsules, tendons, ligaments, dermis, bone, hyaline cartilage, elastic cartilage fibrocartilage, articular cartilage, growth plate cartilage, endothelial cells, meninges, periosteum, perichondrium, erythrocytes, lymphocytes, monocytes, macrophages, microglia, plasma cells, mast cells, dendritic cells, megakaryocytes, osteoclasts, chondroclasts, lymph nodes, tonsils, spleen, kidney, ureter, urinary bladder, heart, testes, ovaries, uterus, etc.

“Pluripotent ectodermal stem cell(s)” are capable of self renewal or differentiation to any particular lineage within the ectodermal germ layer. Pluripotent ectodermal stem-like cells have the ability to commit within the ectodermal lineage from a single cell any time during their life-span. This commitment process necessitates the use of general or specific ectodermal lineage-commitment agents. Pluripotent ectodermal stem-like cells may form any cell type within the neuroectodermal, neural crest, and/or surface ectodermal lineages.

“Pluripotent neuroectodermal stem cell(s)” are capable of self renewal or differentiation to any particular lineage within the neuroectodermal layer. Pluripotent neuroectodermal stem-like cells have the ability to commit within the neuroectodermal lineage from a single cell any time during their life-span. This commitment process necessitates the use of general or specific neuroectodermal lineage-commitment agents. Pluripotent neuroectodermal stem-like cells may form any cell type within the neuroectodermal lineage, including, but not limited to, neurons, oligodendrocytes, astrocytes, ependymal cells, retina, pineal body, posterior pituitary, etc.

“Pluripotent neural crest stem cell(s)” are capable of self renewal or differentiation to any particular lineage within the neural crest layer. Pluripotent neural crest stem-like cells have the ability to commit within the neural crest lineage from a single cell any time during their life-span. This commitment process necessitates the use of general or specific neural crest lineage-commitment agents. Pluripotent neural crest stem-like cells may form any cell type within the neural crest lineage, including, but not limited to, cranial ganglia, sensory ganglia, autonomic ganglia, peripheral nerves, Schwann cells, sensory nerve endings, adrenal medulla, melanocytes, contribute of head mesenchyme, contribute to cervical mesenchyme, contribute to thoracic mesenchyme, contribute to lumbar mesenchyme, contribute to sacral mesenchyme, contribute to coccygeal mesenchyme, heart valves, heart outflow tract (aorta & pulmonary trunk), APUD (amine precursor uptake decarboxylase) system, parafollicular “C” (calcitonin secreting) cells, enterochromaffin cells, etc.

“Pluripotent surface ectodermal stem cell(s)” are capable of self renewal or differentiation to any particular lineage within the surface ectodermal layer. Pluripotent surface ectodermal stem-like cells have the ability to commit within the surface ectodermal lineage from a single cell any time during their life-span. This commitment process necessitates the use of general or specific surface ectodermal lineage-commitment agents. Pluripotent surface ectodermal stem-like cells may form any cell type within the surface ectodermal lineage, including, but not limited to, epidermis, hair, nails, sweat glands, salivary glands, sebaceous glands, mammary glands, anterior pituitary, enamel of teeth, inner ear, lens of the eye, etc.

“Progenitor cell(s)” are lineage-committed, i.e., an individual cell can give rise to progeny limited to a single lineage within their respective germ layers, e.g., liver, thyroid (endoderm), muscle, bone (mesoderm), neuronal, melanocyte, epidermal (ectoderm), etc. They can also be stimulated by particular growth factors to proliferate. If activated to proliferate, progenitor cells have life-spans limited to 50-70 cell doublings before programmed cell senescence and death occurs.

A “clone” or “clonal population” is a population of cells derived from a single cell or common ancestor by mitosis. A “cell line” is a clone of a primary cell that is capable of stable growth in vitro for many generations.

A cell has been “transformed” or “transfected” by exogenous or heterologous DNA when such DNA has been introduced inside the cell. The transforming or transfecting DNA may or may not be integrated (covalently linked) into chromosomal DNA making up the genome of the cell. In prokaryotes, yeast, and mammalian cells for example, the transforming or transfecting DNA may be maintained on an episomal element such as a plasmid. With respect to eukaryotic cells, a stably transformed or transfected cell is one in which the transforming or transfecting DNA has become integrated into a chromosome so that it is inherited by daughter cells through chromosome replication. This stability is demonstrated by the ability of the eukaryotic cell to establish cell lines or clones comprised of a population of daughter cells containing the transforming or transfecting DNA.

The phrase “pharmaceutically acceptable” refers to molecular entities and compositions that are physiologically tolerable and do not typically produce an allergic or similar untoward reaction, such as gastric upset, dizziness and the like, when administered to a human.

The phrase “therapeutically effective amount” is used herein to mean an amount sufficient to prevent, and preferably reduce by at least about 30 percent, more preferably by at least 50 percent, most preferably by at least 90 percent, a clinically significant characteristic of the disease, disorder or condition to be treated.

As used herein, an “enriched population” or “population enriched for” cells having a desired characteristic comprises at least about 50% of cells having the characteristic that defines the population. An enriched population preferably has at least 70%, at least 75%, at least 80%, at least 85%, at least 90%, at least 95%, or at least 99% cells having the particular phenotype, genotype, or other characteristic that defines the population.

As used herein, a “normal cell” is a control cell. In particular, a normal cell is derived from a healthy tissue. Preferably, the normal cell does not include any known mutations that predispose the cell to transformation, and does not display apparent hyperplasia, abnormal or uncontrolled hyperproliferation, or reduced cell death or apoptosis (e.g., a non-cancer cell). In particular embodiments, a “normal cell” is not a naturally occurring, non-disease associated multinucleate cell, such as a myofibril, a macrophage, or bone marrow derived stem-like cells, or a naturally occurring, non-disease associated fused cell such as a gamete. As used herein, a neoplastic cell is a cell that displays apparent hyperplasia or abnormal or uncontrolled hyperproliferation or reduced cell death or apoptosis (e.g., a cancer cell, cells immortalized in culture, a transformed cell).

As used herein, “selecting” is understood as identifying and isolating or enriching for a cell having a desired characteristic. The selected members can be isolated from their original environment and can be pooled. In one embodiment, cells are selected for having undergone fusion. Alternatively, or in addition, selection can be performed based on the expression or the absence of expression of one or more proteins. Protein markers for which cells may be selected include, but are not limited to, CD44, CD24, B38.1, CD2, CD3, CD10, CD14, CD16, CD31, CD45, CD64, CD140b, and ESA. A cell can be selected for being “positive” for a marker, “low” for a marker, or “negative” for a marker, or for being positive, low, or negative for any of a combination of a number of markers. Cells that are positive exhibit detectable levels of a marker. Where the level of a marker in a cell is described as increased or decreased, the level is measured relative to the levels present in a reference cell (e.g., an untreated control cell). Methods for selecting cells are well known and include fluorescence activated cell sorting (FACS) and manual cell selection. The specific method of selection is not a limitation of the instant invention. Selection can be performed based on visual identification of cells having the desire properties, i.e., multinucleate cells. Selection can be performed for cells that may or may not have fused based on the mixing or absence of mixing of detectable cytoplasmic markers or labels (e.g., vital dyes, fluorescent proteins such as green FP and red FP), the amount of nuclear staining with more fluorescence indicative of more nuclei, or the size of cells.

By “population” is meant at least 2 cells. In a preferred embodiment, population is at least 5, 10, 50, 100, 500, 1000, or more cells.

By “isolated” is meant a material that is free to varying degrees from components which normally accompany it as found in its native state. “Isolate” denotes a degree of separation from original source or surroundings. For example, an isolated cell can be removed from an animal and placed in a culture dish or another animal. Isolated is not meant as being removed from all other cells. A polypeptide or nucleic acid is isolated when it is about 80% free, 85% free, 90% free, 95% free from other cellular material, or culture medium when produced by recombinant techniques, or substantially free of chemical precursors or other chemicals when chemically synthesized. An “isolated polypeptide” or “isolated polynucleotide” is, therefore, a substantially purified polypeptide or polynucleotide, respectively.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated. More than one dose may be required for prevention of a disease or condition.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition. More than one dose may be required for prevention of a disease or condition.

By “alteration” is meant a positive or negative alteration. In one embodiment, the alteration is in the expression level or biological activity of a gene or polypeptide as detected by standard art known methods such as those described herein. As used herein, an alteration includes a 10% change in expression levels, preferably a 25% change, more preferably a 40% change, and most preferably a 50% or greater change in expression levels.

As used herein, “obtaining” is understood as purchase, procure, manufacture, or otherwise come into possession of the desired material.

Cells and/or subjects may be treated and/or contacted with one or more anti-neoplastic treatments including, surgery, chemotherapy, radiotherapy, gene therapy, immune therapy or hormonal therapy, or other therapy recommended or proscribed by self or by a health care provider.

The term “subject” includes organisms which are capable of suffering from cancer or other disease of interest who could otherwise benefit from the administration of a compound or composition of the invention, such as human and non-human animals. Preferred human animals include human patients suffering from or prone to suffering from cancer or associated state, as described herein. The term “non-human animals” of the invention includes all vertebrates, e.g., mammals, e.g., rodents, e.g., mice, and non-mammals, such as non-human primates, e.g., sheep, dog, cow, chickens, amphibians, reptiles, etc. A human subject can be referred to as a patient.

In one embodiment, the instant invention pertains to methods for dedifferentiation of somatic cells, e.g., fibroblast cells. In one embodiment, the methods involve contacting the somatic cells with a culture medium comprising BSA (e.g., lipid-rich BSA), for a time and under conditions to allow for the fibroblast to dedifferentiate into a stem cell, e.g., a pluripotent stem cell.

Moreover, the invention provides methods to maintain the somatic cell-derived stem-like cells using serum replacement media supplemented with basic fibroblast growth factor (bFGF). In other embodiments, the media could be supplemented with transforming growth factor, epidermal growth factor, or other fibroblast growth factors.

In certain embodiments of the invention, the methods described above for producing stem-like cells, can be used in combination. For example, serum albumin can be used in combination with other agents, e.g., ions such as silver, e.g., AgNO₃, to produce stem-like cells from somatic cells, e.g., stem-like cells.

In other embodiments of the invention, the methods of the invention result in at least about 5, 10, 15, 20, 25, 30, 35, 40, 45, 50, 55, 60, 65, 70, 75, 80, 85, 90, 95, or 99% of the somatic cells used in the methods of the invention being converted to stem-like cells, e.g., pluripotent stem-like cells.

In another embodiment, the invention provides a mature stem cell. As used herein, the term “mature stem cell” is intended to mean stem-like cells, e.g., pluripotent stem-like cells, comprising mutations acquired by a somatic cell prior to dedifferentiation according to the methods of the invention.

In one aspect, the present invention pertains to the dedifferentiated fibroblasts, i.e., the fibroblast-derived pluripotent stem-like cells. The stem-like cells are stable, i.e., exist for non-transient amounts of time, capable of self-regeneration and capable of differentiation to cells of endodermal, ectodermal and mesodermal lineages.

The pluripotent stem cell of the present invention may be derived from non-human somatic cells or from human somatic cells. In an exemplary embodiment, the pluripotent stem-like cells of the invention are derived from human or non-human fibroblasts.

In one embodiment, the pluripotent stem cell of the present invention is derived from a fibroblast.

As used herein, the term “fibroblast” is intended to mean a mesodermally derived cell from which connective tissue develops.

This invention further relates to cells, particularly pluripotent or progenitor cells, which are derived from fibroblast cells. The cells may be lineage-committed cells, which cells may be committed to the endodermal, ectodermal or mesodermal lineage.

In one embodiment, the present invention relates to pluripotent stem-like cells or populations of such cells derived from fibroblasts which have been transformed or transfected and thereby contain and can express a gene or protein of interest. Thus, this invention includes pluripotent stem-like cells genetically engineered to express a gene or protein of interest. In as much as such genetically engineered stem-like cells can then undergo lineage-commitment, the present invention further encompasses lineage-committed cells, which are derived from a genetically engineered pluripotent stem cell, and which express a gene or protein of interest. The lineage-committed cells may be endodermal, ectodermal or mesodermal lineage-committed cells and may be pluripotent, such as a pluripotent mesenchymal stem cell, or progenitor cells, such as an adipogenic or a myogenic cell.

The invention then relates to methods of producing a genetically engineered pluripotent stem cell derived from a somatic cell, e.g., a fibroblast, comprising the steps of:

transfecting pluripotent stem-like cells with a DNA construct comprising at least one of a marker gene or a gene of interest; selecting for expression of the marker gene or gene of interest in the pluripotent stem-like cells; and culturing the stem-like cells.

The possibilities both diagnostic and therapeutic that are raised by the generation and isolation of the pluripotent stem-like cells of the present invention, derive from the fact that the pluripotent stem-like cells can be generated from readily available somatic cells, e.g., fibroblasts, and are capable of self regeneration on the one hand and of differentiation to cells of endodermal, ectodermal and mesodermal lineages on the other hand, and thus are capable of asymmetric replication. Thus, cells of any of the endodermal, ectodermal and mesodermal lineages can be provided from a single, self-regenerating source of cells obtainable from an animal source even into and through adulthood. As suggested earlier and elaborated further on herein, the present invention contemplates use of the pluripotent stem-like cells, including cells or tissues derived therefrom, for instance, in pharmaceutical intervention, methods and therapy, cell-based therapies, gene therapy, various biological and cellular assays, isolation and assessment of proliferation or lineage-commitment factors, and in varied studies of development and cell differentiation.

The ability to regenerate most human tissues damaged or lost due to trauma or disease is substantially diminished in adults. Every year millions of Americans suffer tissue loss or end-stage organ failure. Tissue loss may result from acute injuries as well as surgical interventions, i.e., amputation, tissue debridement, and surgical extirpations with respect to cancer, traumatic tissue injury, congenital malformations, vascular compromise, elective surgeries, etc. Options such as tissue transplantation and surgical intervention are severely limited by a critical donor shortage and possible long term morbidity. Three general strategies for tissue engineering have been adopted for the creation of new tissue: (1). Isolated cells or cell substitutes applied to the area of tissue deficiency or compromise. (2). Cells placed on or within matrices, in either closed or open systems. (3). Tissue-inducing substances, that rely on growth factors (including proliferation factors or lineage-commitment factors) to regulate specific cells to a committed pattern of growth resulting in tissue regeneration, and methods to deliver these substances to their targets.

A wide variety of transplants, congenital malformations, elective surgeries, diseases, and genetic disorders have the potential for treatment with the pluripotent stem-like cells of the present invention, including cells or tissues derived therefrom, alone or in combination with proliferation factors, lineage-commitment factors, or genes or proteins of interest. Preferred treatment methods include the treatment of tissue loss where the object is to provide cells directly for transplantation whereupon the tissue can be regenerated in vivo, recreate the missing tissue in vitro and then provide the tissue, or providing sufficient numbers of cells suitable for transfection or transformation for ex vivo or in vivo gene therapy.

As described above, the cells of the present invention have the capacity to differentiate into cells of any of the ectodermal, mesodermal, and endodermal lineage. The capacity for such differentiation in vitro (in culture) and in vivo, even to correct defects and function in vivo is readily understood by those of skill in the art. Thus, the cells of the present invention may be utilized in transplantation, cell replacement therapy, tissue regeneration, gene therapy, organ replacement and cell therapies wherein cells, tissues, organs of mesodermal, ectodermal and/or endodermal origin are derived in vivo, ex vivo or in vitro. Endoderm cell, tissue or organ therapy and/or regeneration and/or therapy utilizing the stem-like cells of the invention or their derived differentiated or progenitor cells may useful as the cell source for epithelial linings of the respiratory passages and gastrointestinal tract, the pharynx, esophagus, stomach, intestine and to many associated glands, including salivary glands, liver, pancreas and lungs. In particular and as non-limiting examples, liver transplantation and pancreas cell replacement for diabetes is thereby contemplated. Mesoderm cell, tissue or organ therapy and/or regeneration and/or therapy utilizing the pluripotent stem-like cells of the invention or their derived differentiated or progenitor cells may useful as the cell source for smooth muscular coats, connective tissues, and vessels associated with tissues and organs and for replacement/therapy of the cardiovascular system, heart, cardiac muscle, cardiac vessels, other vessels, blood cells, bone marrow, the skeleton, striated muscles, and the reproductive and excretory organs. Ectoderm cell, tissue or organ therapy and/or regeneration and/or therapy utilizing the pluripotent stem-like cells of the invention or their derived differentiated or progenitor cells may useful as the cell source for the epidermis (epidermal layer of the skin), the sense organs, and the entire nervous system, including brain, spinal cord, and all the outlying components of the nervous system. A significant benefit of the pluripotent stem-like cells of the present invention are their potential for self-regeneration prior to commitment to any particular tissue lineage (ectodermal, endodermal or mesodermal) and then further proliferation once committed. Moreover, stem-like cells of the instant invention can be produced from somatic cells of the patient in need of treatment. These proliferative and differentiative attributes are very important and useful when limited amounts of appropriate cells and tissue are available for transplantation.

In a further embodiment, the present invention relates to certain therapeutic methods which would be based upon the activity of the pluripotent stem-like cells of the present invention, including cells or tissues derived therefrom, or upon agents or other drugs determined to act on any such cells or tissues, including proliferation factors and lineage-commitment factors. One exemplary therapeutic method is associated with the prevention or modulation of the manifestations of conditions causally related to or following from the lack or insufficiency of cells of a particular lineage, and comprises administering the pluripotent stem-like cells of the present invention, including cells or tissues derived therefrom, either individually or in mixture with proliferation factors or lineage-commitment factors in an amount effective to prevent the development or progression of those conditions in the host.

In a further and particular aspect the present invention includes therapeutic methods, including transplantation of the pluripotent stem-like cells of the present invention, including lineage-uncommitted populations of cells, lineage-committed populations of cells, tissues and organs derived therefrom, in treatment or alleviation of conditions, diseases, disorders, cellular debilitations or deficiencies which would benefit from such therapy. These methods include the replacement or replenishment of cells, tissues or organs. Such replacement or replenishment may be accomplished by transplantation of the pluripotent stem-like cells of the present invention or by transplantation of lineage-uncommitted populations of cells, lineage-committed populations of cells, tissues or organs derived therefrom.

Thus, the present invention includes a method of transplanting pluripotent stem-like cells in a host comprising the step of introducing into the host the pluripotent stem-like cells of the present invention.

In a further aspect this invention provides a method of providing a host with purified pluripotent stem-like cells comprising the step of introducing into the host the pluripotent stem-like cells of the present invention. In one aspect, the pluripotent stem-like cells administered to a host are derived from the subject's own somatic cells, e.g., fibroblast cells.

In a still further aspect, this invention includes a method of in vivo administration of a protein or gene of interest comprising the step of transfecting the pluripotent stem-like cells of the present invention with a vector comprising DNA or RNA which expresses a protein or gene of interest.

The present invention provides a method of preventing and/or treating cellular debilitations, derangements and/or dysfunctions and/or other disease states in mammals, comprising administering to a mammal a therapeutically effective amount of pluripotent stem-like cells.

In a further aspect, the present invention provides a method of preventing and/or treating cellular debilitations, derangements and/or dysfunctions and/or other disease states in mammals, comprising administering to a mammal a therapeutically effective amount of a endodermal, ectodermal or mesodermal lineage-committed cell derived from the pluripotent stem-like cells of the present invention.

The therapeutic method generally referred to herein could include the method for the treatment of various pathologies or other cellular dysfunctions and derangements by the administration of pharmaceutical compositions that may comprise proliferation factors or lineage-commitment factors, alone or in combination with the pluripotent stem-like cells of the present invention, or cells or tissues derived therefrom, or other similarly effective agents, drugs or compounds identified for instance by a toxicity or drug screening assay prepared and used in accordance with a further aspect of the present invention.

Also, antibodies including both polyclonal and monoclonal antibodies that recognize the pluripotent stem-like cells of the present invention, including cells and/or tissues derived therefrom, and agents, factors or drugs that modulate the proliferation or commitment of the pluripotent stem-like cells of the present invention, including cells and/or tissues derived therefrom, may possess certain diagnostic or therapeutic applications and may for example, be utilized for the purpose of correction, alleviation, detecting and/or measuring conditions such as cellular debilitations, cellular deficiencies or the like. For example, the pluripotent stem-like cells of the present invention, including cells and/or tissues derived therefrom, may be used to produce both polyclonal and monoclonal antibodies to themselves in a variety of cellular media, by known techniques such as the hybridoma technique utilizing, for example, fused mouse spleen lymphocytes and myeloma cells. Likewise, agents, factors or drugs that modulate, for instance, the proliferation or commitment of the cells of the invention may be discovered, identified or synthesized, and may be used in diagnostic and/or therapeutic protocols.

The general methodology for making monoclonal antibodies by hybridomas is well known. Immortal, antibody-producing cell lines can also be created by techniques other than fusion, such as direct transformation of B lymphocytes with oncogenic DNA, or transfection with Epstein-Barr virus. See, e.g., M. Schreier et al., “Hybridoma Techniques” (1980); Hammerling et al., “Monoclonal Antibodies And T-cell Hybridomas” (1981); Kennett et al., “Monoclonal Antibodies” (1980); see also U.S. Pat. Nos. 4,341,761; 4,399,121; 4,427,783; 4,444,887; 4,451,570; 4,466,917; 4,472,500; 4,491,632; 4,493,890.

Panels of monoclonal antibodies produced against the pluripotent stem-like cells, including cells or tissues derived therefrom, or against proliferation or lineage-commitment factors that act thereupon, can be screened for various properties; i.e., isotype, epitope, affinity, etc. Of particular interest are monoclonal antibodies that neutralize the activity of the proliferation or lineage-commitment factors. Such monoclonals can be readily identified in activity assays, including lineage commitment or proliferation assays as contemplated or described herein. High affinity antibodies are also useful when immunoaffinity-based purification or isolation or identification of the pluripotent stem-like cells, including cells or tissues therefrom, or of proliferation or lineage-commitment factors is sought.

Preferably, the antibody used in the diagnostic or therapeutic methods of this invention is an affinity purified polyclonal antibody. More preferably, the antibody is a monoclonal antibody (mAb). In addition, it is preferable for the antibody molecules used herein to be in the form of Fab, Fab′, F(ab′)₂ or F(v) portions of whole antibody molecules.

The diagnostic method of the present invention may, for instance, comprise examining a cellular sample or medium by means of an assay including an effective amount of an antibody recognizing the stem-like cells of the present invention, including cells or tissues derived therefrom, such as an anti-pluripotent stem cell antibody, preferably an affinity-purified polyclonal antibody, and more preferably a mAb. In addition, it is preferable for the antibody molecules used herein to be in the form of Fab, Fab′, F(ab′)₂ or F(v) portions or whole antibody molecules. As previously discussed, patients capable of benefiting from this method include those suffering from cellular debilitations, organ failure, tissue loss, tissue damage, congenital malformations, cancer, or other diseases or debilitations. Methods for isolating the antibodies and for determining and optimizing the ability of antibodies to assist in the isolation, purification, examination or modulation of the target cells or factors are all well-known in the art.

The present invention further contemplates therapeutic compositions useful in practicing the therapeutic methods of this invention. A subject therapeutic composition includes, in admixture, a pharmaceutically acceptable excipient (carrier) or media and one or more of the pluripotent stem-like cells of the present invention, including cells or tissues derived therefrom, alone or in combination with proliferation factors or lineage-commitment factors, as described herein as an active ingredient.

The stem-like cells of the present invention, including cells or tissues derived therefrom, alone or in combination with proliferation factors or lineage-commitment factors, may be prepared in pharmaceutical compositions, with a suitable carrier and at a strength effective for administration by various means to a patient experiencing cellular or tissue loss or deficiency.

In one embodiment, the invention provides for the treatment of diseases and disorders.

In various embodiments, the stem-like cells of the invention are driven to differentiate in vitro using any agent that promotes the differentiation of a stem cell. Exemplary agents include, but are not limited to, any one or more of activin A, adrenomedullin, acidic FGF, basic fibroblast growth factor, angiogenin, angiopoietin-1, angiopoietin-2, angiopoietin-3, angiopoietin-4, angiostatin, angiotropin, angiotensin-2, bone morphogenic protein 1, 2, or 3, cadherin, collagen, colony stimulating factor (CSF), endothelial cell-derived growth factor, endoglin, endothelin, endostatin, endothelial cell growth inhibitor, endothelial cell-viability maintaining factor, ephrins, erythropoietin, fibronectin, granulocyte macrophage colony stimulating factor (GM-CSF), hepatocyte growth factor, human growth hormone, IFN-gamma, LIF, insulin, insulin-like growth factor-1 or -2 (IGF), interleukin (IL)-1 or 8, platelet derived endothelial growth factor (PDGF), retinoic acid, trans-retinoic acid, stem cell factor (SCF), TNF-alpha, TGF-beta, VEGF-A, VEGF-B, VEGF-C, VEGF-D, VEGF-E, VEGF, and VEGF164. Agents comprising growth factors are known in the art to differentiate stem-like cells. Such agents are expected to be similarly useful for inducing the differentiation of a stem-like cell. In an embodiment, such agents are used to promote differentiation of tumorogenic cells to increase susceptibility to chemotherapeutic agents.

Differentiated cells are identified as differentiated, for example, by the expression of markers, by cellular morphology, or by the ability to form a particular cell type (e.g., ectodermal cell, mesodermal cell, endodermal cell, adipocyte, myocyte, neuron). Those skilled in the art can readily determine the percentage of differentiated cells in a population using various well-known methods, such as fluorescence activated cell sorting (FACS). Preferable ranges of purity in populations comprising differentiated cells are about 50 to about 55%, about 55 to about 60%, and about 65 to about 70%. More preferably the purity is about 70 to about 75%, about 75 to about 80%, about 80 to about 85%; and still more preferably the purity is about 85 to about 90%, about 90 to about 95%, and about 95 to about 100%. Purity cells or their progenitors can be determined according to the marker profile within a population. Dosages can be readily adjusted by those skilled in the art (e.g., a decrease in purity may require an increase in dosage).

Differentiated cells of the invention can be provided directly to a tissue or organ of interest (e.g., by direct injection). In one embodiment, cells of the invention are provided to a site where an increase in the number of cells is desired, for example, due to disease, damage, injury, or excess cell death. Alternatively, cells of the invention can be provided indirectly to a tissue or organ of interest, for example, by administration into the circulatory system. If desired, the cells are delivered to a portion of the circulatory system that supplies the tissue or organ to be repaired or regenerated.

Advantageously, cells of the invention engraft within the tissue or organ. If desired, expansion and differentiation agents can be provided prior to, during or after administration of the cells to increase, maintain, or enhance production or differentiation of the cells in vivo. Compositions of the invention include pharmaceutical compositions comprising differentiated cells or their progenitors and a pharmaceutically acceptable carrier. Administration can be autologous or heterologous. For example, cells obtained from one subject, can be administered to the same subject or a different, compatible subject. Methods for administering cells are known in the art, and include, but are not limited to, catheter administration, systemic injection, localized injection, intravenous injection, intramuscular, intracardiac injection or parenteral administration. When administering a therapeutic composition of the present invention (e.g., a pharmaceutical composition), it will generally be formulated in a unit dosage injectable form (solution, suspension, emulsion).

It is a still further object of the present invention to provide pharmaceutical compositions for use in therapeutic methods which comprise or are based upon the pluripotent stem-like cells of the present invention, including lineage-uncommitted populations of cells, lineage-committed populations of cells, tissues and organs derived therefrom, along with a pharmaceutically acceptable carrier or media. Also contemplated are pharmaceutical compositions comprising proliferation factors or lineage commitment factors that act on or modulate the pluripotent stem-like cells of the present invention and/or the cells, tissues and organs derived therefrom, along with a pharmaceutically acceptable carrier or media. The pharmaceutical compositions of proliferation factors or lineage commitment factors may further comprise the pluripotent stem-like cells of the present invention, or cells, tissues or organs derived therefrom.

The pharmaceutical compositions of the present invention may comprise the pluripotent stem-like cells of the present invention, or cells, tissues or organs derived therefrom, alone or in a polymeric carrier or extracellular matrix.

Compositions of the invention (e.g., cells in a suitable vehicle) can be provided directly to an organ of interest, such as an organ having a deficiency in cell number as a result of injury or disease. Alternatively, compositions can be provided indirectly to the organ of interest, for example, by administration into the circulatory system. Compositions can be administered to subjects in need thereof by a variety of administration routes. Methods of administration, generally speaking, may be practiced using any mode of administration that is medically acceptable, meaning any mode that produces effective levels of the active compounds without causing clinically unacceptable adverse effects. Such modes of administration include intramuscular, intra-cardiac, oral, rectal, topical, intraocular, buccal, intravaginal, intracisternal, intracerebroventricular, intratracheal, nasal, transdermal, within/on implants, e.g., fibers such as collagen, osmotic pumps, or grafts comprising differentiated cells, etc., or parenteral routes. The term “parenteral” includes subcutaneous, intravenous, intramuscular, intraperitoneal, intragonadal or infusion. A particular method of administration involves coating, embedding or derivatizing fibers, such as collagen fibers, protein polymers, etc. with therapeutic proteins. Other useful approaches are described in Otto, D. et al., J. Neurosci. Res. 22: 83 and in Otto, D. and Unsicker, K. J. Neurosci. 10: 1912.

In one approach, stem-like cells derived from cultures of the invention are implanted into a host. The transplantation can be autologous, such that the donor of the cells is the recipient of the transplanted cells; or the transplantation can be heterologous, such that the donor of the cells is not the recipient of the transplanted cells. Once transferred into a host, the re-stem-like cells are engrafted, such that they assume the function and architecture of the native host tissue.

In another approach, stem-like cells derived from the stem-like cells of the invention are implanted into a host. The transplantation can be autologous, such that the donor of the cells is the recipient of the transplanted cells; or the transplantation can be heterologous, such that the donor of the cells is not the recipient of the transplanted cells. The stem-like cells are then engrafted, such that they assume the function and architecture of the native host tissue.

Stem-like cells and the progenitors thereof can be cultured, treated with agents and/or administered in the presence of polymer scaffolds. If desired, agents described herein are incorporated into the polymer scaffold to promote cell survival, proliferation, enhance maintenance of a cellular phenotype. Polymer scaffolds are designed to optimize gas, nutrient, and waste exchange by diffusion. Polymer scaffolds can comprise, for example, a porous, non-woven array of fibers. The polymer scaffold can be shaped to maximize surface area, to allow adequate diffusion of nutrients and growth factors to the cells. Taking these parameters into consideration, one of skill in the art could configure a polymer scaffold having sufficient surface area for the cells to be nourished by diffusion until new blood vessels interdigitate the implanted engineered-tissue using methods known in the art. Polymer scaffolds can comprise a fibrillar structure. The fibers can be round, scalloped, flattened, star-shaped, solitary or entwined with other fibers. Branching fibers can be used, increasing surface area proportionately to volume.

Unless otherwise specified, the term “polymer” includes polymers and monomers that can be polymerized or adhered to form an integral unit. The polymer can be non-biodegradable or biodegradable, typically via hydrolysis or enzymatic cleavage. The term “biodegradable” refers to materials that are bioresorbable and/or degrade and/or break down by mechanical degradation upon interaction with a physiological environment into components that are metabolizable or excretable, over a period of time from minutes to three years, preferably less than one year, while maintaining the requisite structural integrity. As used in reference to polymers, the term “degrade” refers to cleavage of the polymer chain, such that the molecular weight stays approximately constant at the oligomer level and particles of polymer remain following degradation.

Materials suitable for polymer scaffold fabrication include polylactic acid (PLA), poly-L-lactic acid (PLLA), poly-D-lactic acid (PDLA), polyglycolide, polyglycolic acid (PGA), polylactide-co-glycolide (PLGA), polydioxanone, polygluconate, polylactic acid-polyethylene oxide copolymers, modified cellulose, collagen, polyhydroxybutyrate, polyhydroxpriopionic acid, polyphosphoester, poly(alpha-hydroxy acid), polycaprolactone, polycarbonates, polyamides, polyanhydrides, polyamino acids, polyorthoesters, polyacetals, polycyanoacrylates, degradable urethanes, aliphatic polyester polyacrylates, polymethacrylate, acyl substituted cellulose acetates, non-degradable polyurethanes, polystyrenes, polyvinyl chloride, polyvinyl flouride, polyvinyl imidazole, chlorosulphonated polyolifins, polyethylene oxide, polyvinyl alcohol, Teflon®, nylon silicon, and shape memory materials, such as poly(styrene-block-butadiene), polynorbornene, hydrogels, metallic alloys, and oligo(ε-caprolactone)diol as switching segment/oligo(p-dioxyanone)diol as physical crosslink. Other suitable polymers can be obtained by reference to The Polymer Handbook, 3rd edition (Wiley, N.Y., 1989).

This invention also provides pharmaceutical compositions for the treatment of cellular debilitation, derangement and/or dysfunction in mammals, comprising: a therapeutically effective amount of the pluripotent stem-like cells of the present invention; and a pharmaceutically acceptable medium or carrier.

Pharmaceutical compositions of the present invention also include compositions comprising endodermal, ectodermal or mesodermal lineage-committed cell(s) derived from the pluripotent stem-like cells of the present invention, and a pharmaceutically acceptable medium or carrier. Any such pharmaceutical compositions may further comprise a proliferation factor or lineage-commitment factor.

A variety of administrative techniques may be utilized, among them parenteral techniques such as subcutaneous, intravenous and intraperitoneal injections, catheterizations and the like. The therapeutic factor-containing compositions are conventionally administered intravenously, as by injection of a unit dose, for example. Average quantities of the stem-like cells or cells may vary and in particular should be based upon the recommendations and prescription of a qualified physician or veterinarian.

The preparation of cellular or tissue-based therapeutic compositions as active ingredients is well understood in the art. Such compositions may be formulated in a pharmaceutically acceptable media. The cells may be in solution or embedded in a matrix.

The preparation of therapeutic compositions with factors, including growth, proliferation or lineage-commitment factors, (such as for instance human growth hormone) as active ingredients is well understood in the art. The active therapeutic ingredient is often mixed with excipients or media which are pharmaceutically acceptable and compatible with the active ingredient. In addition, if desired, the composition can contain minor amounts of auxiliary substances such as wetting or emulsifying agents, pH buffering agents which enhance the effectiveness of the active ingredient.

A factor can be formulated into the therapeutic composition as neutralized pharmaceutically acceptable salt forms. Pharmaceutically acceptable salts include the acid addition salts (formed with the free amino groups of the polypeptide or antibody molecule) and which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

The term “unit dose” when used in reference to a therapeutic composition of the present invention refers to physically discrete units suitable as unitary dosage for humans, each unit containing a predetermined quantity of active material calculated to produce the desired therapeutic effect in association with the required diluent; i.e., carrier, media, or vehicle.

The compositions are administered in a manner compatible with the dosage formulation, and in a therapeutically effective amount. The quantity to be administered depends, for instance, on the subject and debilitation to be treated, capacity of the subject's organ, cellular and immune system to utilize the active ingredient, and the nature of the cell or tissue therapy, etc. Precise amounts of active ingredient required to be administered depend on the judgment of the practitioner and are peculiar to each individual. However, suitable dosages of a factor may range from about 0.1 to 20, preferably about 0.5 to about 10, and more preferably one to several, milligrams of active ingredient per kilogram body weight of individual per day and depend on the route of administration. Suitable regimes for initial administration and follow on administration are also variable, but can include an initial administration followed by repeated doses at one or more hour intervals by a subsequent injection or other administration. Alternatively, continuous intravenous infusion sufficient to maintain concentrations of ten nanomolar to ten micromolar in the blood are contemplated.

One consideration concerning the therapeutic use of differentiated cells of the invention or their progenitors is the quantity of cells necessary to achieve an optimal effect. In general, doses ranging from 1 to 4×10⁷ cells may be used. However, different scenarios may require optimization of the amount of cells injected into a tissue of interest. Thus, the quantity of cells to be administered will vary for the subject being treated. In a preferred embodiment, between 10⁴ to 10⁸, more preferably 10⁵ to 10⁷, and still more preferably, 1, 2, 3, 4, 5, 6, 7×10⁷ stem-like cells of the invention can be administered to a human subject.

Fewer cells can be administered directly a tissue where an increase in cell number is desirable. Preferably, between 10² to 10⁶, more preferably 10³ to 10⁵, and still more preferably, 10⁴ stem-like cells or their progenitors can be administered to a human subject. However, the precise determination of what would be considered an effective dose may be based on factors individual to each subject, including their size, age, sex, weight, and condition of the particular subject. As few as 100-1000 cells can be administered for certain desired applications among selected patients. Therefore, dosages can be readily ascertained by those skilled in the art from this disclosure and the knowledge in the art.

The skilled artisan can readily determine the amount of cells and optional additives, vehicles, and/or carrier in compositions and to be administered in methods of the invention. Typically, any additives (in addition to the active stem cell(s) and/or agent(s)) are present in an amount of 0.001 to 50% (weight) solution in phosphate buffered saline, and the active ingredient is present in the order of micrograms to milligrams, such as about 0.0001 to about 5 wt %, preferably about 0.0001 to about 1 wt %, still more preferably about 0.0001 to about 0.05 wt % or about 0.001 to about 20 wt %, preferably about 0.01 to about 10 wt %, and still more preferably about 0.05 to about 5 wt %. Of course, for any composition to be administered to an animal or human, and for any particular method of administration, it is preferred to determine therefore: toxicity, such as by determining the lethal dose (LD) and LD₅₀ in a suitable animal model e.g., rodent such as mouse; and, the dosage of the composition(s), concentration of components therein and timing of administering the composition(s), which elicit a suitable response. Such determinations do not require undue experimentation from the knowledge of the skilled artisan, this disclosure and the documents cited herein. And, the time for sequential administrations can be ascertained without undue experimentation.

If desired, cells of the invention are delivered in combination with (prior to, concurrent with, or following the delivery of) agents that increase survival, increase proliferation, enhance differentiation, and/or promote maintenance of a differentiated cellular phenotype. In vitro and ex vivo applications of the invention involve the culture of stem-like cells or their progenitors with a selected agent to achieve a desired result. Cultures of cells (from the same individual and from different individuals) can be treated with expansion agents prior to, during, or following differentiation to increase the number of differentiated cells. Similarly, differentiation agents of interest can be used to generate a differentiated cell from a tumor-initating cell. Stem-like cells can then be used for a variety of therapeutic applications (e.g., tissue or organ repair, regeneration, treatment of an ischemic tissue, or treatment of myocardial infarction). If desired, stem-like cells of the invention are delivered in combination with other factors that promote cell survival, differentiation, or engraftment. Such factors, include but are not limited to nutrients, growth factors, agents that induce differentiation, products of secretion, immunomodulators, inhibitors of inflammation, regression factors, hormones, or other biologically active compounds.

The present invention also relates to a variety of diagnostic applications, including methods for detecting the presence of proliferation factors or particular lineage-commitment factors, by reference to their ability to elicit proliferation or particular lineage commitment of pluripotent stem-like cells, including cells or tissues derived therefrom. The diagnostic utility of the pluripotent stem-like cells of the present invention extends to the use of such cells in assays to screen for proliferation factors or particular lineage-commitment factors, by reference to their ability to elicit proliferation or particular lineage commitment of pluripotent stem-like cells, including cells or tissues derived therefrom. Such assays may be used, for instance, in characterizing a known factor, identifying a new factor, or in cloning a new or known factor by isolation of and determination of its nucleic acid and/or protein sequence.

The presence of pluripotent stem-like cells can be ascertained by the usual immunological procedures applicable to such determinations. A number of useful procedures are known.

The invention includes an assay system for screening of potential agents, compounds or drugs effective to modulate the proliferation or lineage-commitment of the pluripotent stem-like cells of the present invention, including cells or tissues derived therefrom. These assays may also be utilized in cloning a gene or polypeptide sequence for a factor, by virtue of the factors known or presumed activity or capability with respect to the pluripotent stem-like cells of the present invention, including cells or tissues derived therefrom.

The assay system could be adapted to identify drugs or other entities that are capable of modulating the pluripotent stem-like cells of the present invention, either in vitro or in vivo. Such an assay would be useful in the development of agents, factors or drugs that would be specific in modulating the pluripotent stem-like cells to, for instance, proliferate or to commit to a particular lineage or cell type. For example, such drugs might be used to facilitate cellular or tissue transplantation therapy.

The present invention contemplates methods for detecting the presence or activity of an agent which is a lineage-commitment factor comprising the steps of:

Contacting the pluripotent stem-like cells of the present invention with a sample suspected of containing an agent which is a lineage-commitment factor; and determining the lineage of the so contacted cells by morphology, mRNA expression, antigen expression or other means; wherein the lineage of the contacted cells indicates the presence or activity of a lineage-commitment factor in said sample.

The present invention also relates to methods of testing the ability of an agent, compound or factor to modulate the lineage-commitment of a lineage uncommitted cell which comprises culturing the pluripotent stem-like cells of the present invention in a growth medium which maintains the stem-like cells as lineage uncommitted cells; adding the agent, compound or factor under test; and determining the lineage of the so contacted cells by morphology, mRNA expression, antigen expression or other means.

In a further such aspect, the present invention relates to an assay system for screening agents, compounds or factors for the ability to modulate the lineage-commitment of a lineage uncommitted cell, comprising: culturing the pluripotent stem-like cells of the present invention in a growth medium which maintains the stem-like cells as lineage uncommitted cells; adding the agent, compound or factor under test; and determining the lineage of the so contacted cells by morphology, mRNA expression, antigen expression or other means.

The invention also relates to a method for detecting the presence or activity of an agent which is a proliferation factor comprising the steps of: contacting the pluripotent stem-like cells of the present invention with a sample suspected of containing an agent which is a proliferation factor; and determining the proliferation and lineage of the so contacted cells by morphology, mRNA expression, antigen expression or other means; wherein the proliferation of the contacted cells without lineage commitment indicates the presence or activity of a proliferation factor in the sample.

The invention further relates to an assay system for screening agents, compounds or factors for the ability to modulate the proliferation of a lineage uncommitted cell, comprising: culturing the pluripotent stem-like cells of the present invention in a growth medium which maintains the stem-like cells as lineage uncommitted cells; adding the agent, compound or factor under test; and

determining the proliferation and lineage of the contacted cells.

In a further embodiment of this invention kits are provided. In one aspect the kit comprises an agent, e.g., aracahdonic acid or serum albumin, that has the ability to convert a somatic cell, e.g., a fibroblast, into a stem-like cell. The kit may further comprise lineage commitment factors for committing the produced stem-like cells to a specific lineage.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

### EXAMPLES

It should be appreciated that the invention should not be construed to be limited to the examples that are now described; rather, the invention should be construed to include any and all applications provided herein and all equivalent variations within the skill of the ordinary artisan.

### Methods

Mouse embryonic skin fibroblast cells (MEFs) derived form 13.5 day mouse embryos and adult mouse primary dermal fibroblasts derived from adult mouse skin were cultured in DMEM Dulbecco's modified Eagle's medium (DMEM) with 10% Fetal Bovine Serum (FBS) at 37° C., 10% CO2. After the cells were grown to confluence, the cells were trypsinized, then suspended in SR-containing medium or basic medium without serum. About 10⁵ mouse skin fibroblast cells were transferred to 3 ml SR-containing medium in a 6 well plate. SR-containing medium contained basic medium that included DMEM/F12 (Invitrogen 11330-032), 1× non-Essential Amino Acids (Invitrogen 11140-050), 1× L-Glutamine (Invitrogen 25030-018), 0.1 μM β-mercaptoethanol and 20% Knockout Serum Replacer (Invitrogen 10828-028). 4 ng/mL bFGF (R&D System Cat# 233-FB) and Leukemia inhibitory factor Lif (ESGRO®, Chemicon ESG1107) were added to the SR-containing medium to prevent stem-like cells from differentiating.

To reconstitute the SR, the ingredients were prepared according to WO 98/30679 (Price et al.). Serum-containing media in which DMEM was supplemented with 15% ES-quality FBS, 1× NEAA (non-essential amino acids), 1× GlutaMax, 100 U/mL penicillin streptomycin, 0.1 μM β-mercaptoethanol, and 50 μM recombinant Leukemia inhibitory factor (LIF, Chemicon) were added to mouse ES and iES cell culture.

Protocol for Conversion of Fibroblasts to Induced Pluripotent Stem (iPS) Cells

Conversion of fibroblast cells to induced pluripotent stem-like cells can be achieved by culturing a mouse embryonic skin fibroblast (MEF), mouse adult skin fibroblast (MAF) or human skin fibroblast cell in DMEM media having 10% fetal bovine serum (FBS) until the cells are confluent. Serum-replacement (SR)-containing media and 0.25% trypsin/EDTA are then warmed (e.g., at 37° C. in a water bath). Media is aspirated from the fibroblast cell culture plate, cells are washed with 1×PBS, and trypsinized via addition of 2 mls of trypsin/EDTA for one minute, followed by removal of all the trypsin solution. 2 mls of the SR human ES medium or DMEM/F12 medium is then added to the cells (without FBS), and cells are resuspended in the medium using a 5 ml pipet, pipeting up and down until no large clumps remain. About 10⁵ cells/well are then added to a six well plate, with each well containing 3 mls of SR human ES medium. The cells are then mixed in this media. Cells are then placed in a warm incubator (e.g., 37° C. at 5% or 10% CO2). The cells are passaged every 3 days for a total of five or more passages. Ingredients of SR human stem cell culture medium are: 400 ml DMEM/F12 (Invitrogen 11330-032), 5 ml non-Essential Amino Acids (Invitrogen 11140-050), 2.5 ml L-Glutamine (Invitrogen 25030-018), 0.1 μM β-mercaptoethanol (Sigma 7522, add 3.5 ul) and 100 ml Knockout Serum Replacer (Invitrogen 10828-028). Optionally, 4 ng/mL bFGF (R&D System Cat# 233-FB) is present. To make FGF stock, 1 ml of DMEM/F12 was added to dissolve 1 vial of 10 ug of FGF, with this solution aliquotted into five vials, then stored at −20° C. bFGF needed to be added freshly to the medium every time the method was performed. Lif was also needed for mouse cell cultures. Formulation of the SR component of serum-free medium also contained AgNO₃ and other trace elements. For AgNO₃-containing media, 1000× AgNO₃ was added to the medium to a final concentration of 0.0001 mg/L.

### Example 1

### SR-Containing Serum Free Medium Promoted Dedifferentiation of Skin Fibroblast Cells into Stem Cell-Like Cells

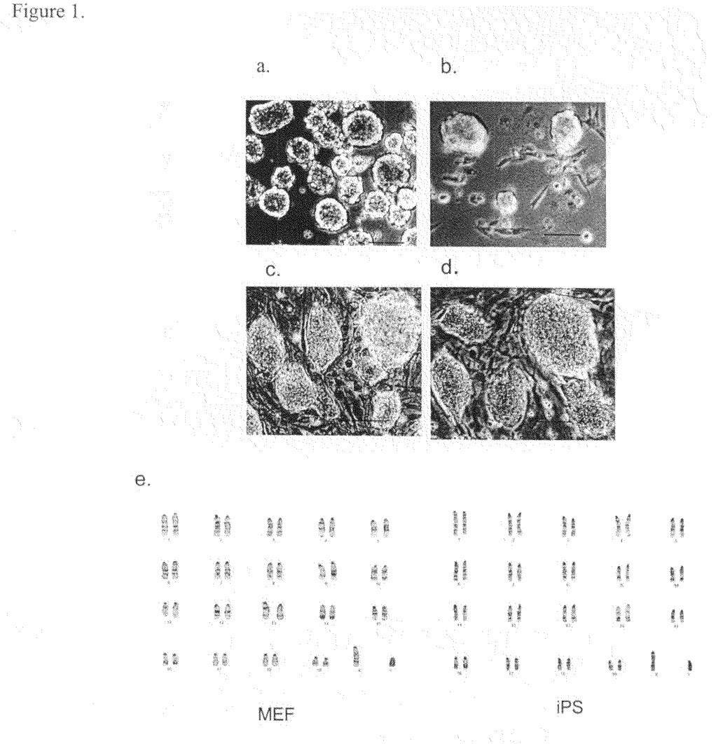

To investigate the possibility that serum replacement (SR)-containing medium can promote the reprogramming and dedifferentiation of skin fibroblast cells into pluripotent stem-like cells, mouse skin fibroblast cells were cultured in fibroblast growth medium, and were then transferred by trypsinization to plates holding SR-containing medium. Within a few hours, the SR-containing medium caused the majority of the transferred cells to become rounded in shape. Twenty-four hours post-transfer, a majority of cells became small, round, bright-edged granulated cells (FIG. 1a). At three days post-transfer, some of the granulated cells grew into large, stem cell-like colonies and attached to the bottom of the plates (FIG. 1b). These ES-like cells could be picked or passaged as a whole plate in SR-containing medium for an additional 5 or more passages, allowing for establishment of a cell line. The established cell lines were able to be cultured in either SR-containing or serum-containing medium with or without feeder cells (FIGS. 1c and d). Addition of bFGF (4 ng/ml) and Leukemic inhibitor factors (Lif) to the SR-containing medium was observed to promote division of the ES-like cells, while preventing differentiation of these cells. Comparison of the karyotypes of P1 and P5 passaged ES-like cells and fibroblast cells revealed that no major translocation, amplification or other chromosomal changes were observed to have occurred during or after reprogramming (FIG. 1e).

### Example 2

### Lipid-Rich BSA in the SR Medium was Essential for Reprogramming Fibroblasts into Stem Cell-Like Cells

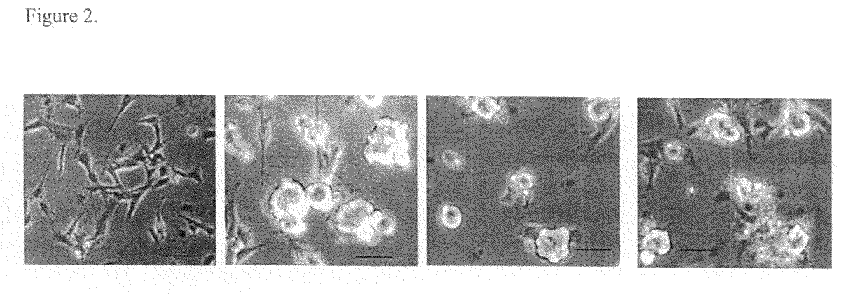

SR-containing ES media consisted of a combination of basal media containing DMEM/F12, 1× non-Essential Amino Acids, 1× L-Glutamine, 0.1 μM β-mercaptoethanol and 20% Serum Replacer (SR). To further examine whether SR was the key component responsible for promoting the reprogramming of skin fibroblasts into ES-like cells, skin fibroblast cells were transferred by trypsinization to basal medium with or without SR. Results of these experiments demonstrated that SR was the critical component for promoting reprogramming of fibroblast cells into ES-like cells (FIG. 2a).

The formulation of SR that was present in serum-free medium comprised ingredients such as thiamine, reduced glutathiones, ascorbic acid-2-PO4, transferrin, insulin, and lipid-rich BSA (AlbuMax I, Invitrogen; Costagliola and Agrosi. Curr. Med. Res. Opin. 21:1235). To identify the component(s) of SR that possessed the ability to promote dedifferentiation of skin fibroblasts into ES-like cells, SR components were otherwise reconstituted while eliminating individual supplements such as thiamine, reduced glutathiones, ascorbic acid-2-PO4, transferrin, insulin, and lipid-rich BSA, respectively.

### Example 3

### SR Induced Fibroblast Cell Reprogramming by Increasing Ca²⁺ Influx, Increasing Fibroblast Growth Factor Receptor 3 Expression and Causing Demethylation of the Oct4 Promoter

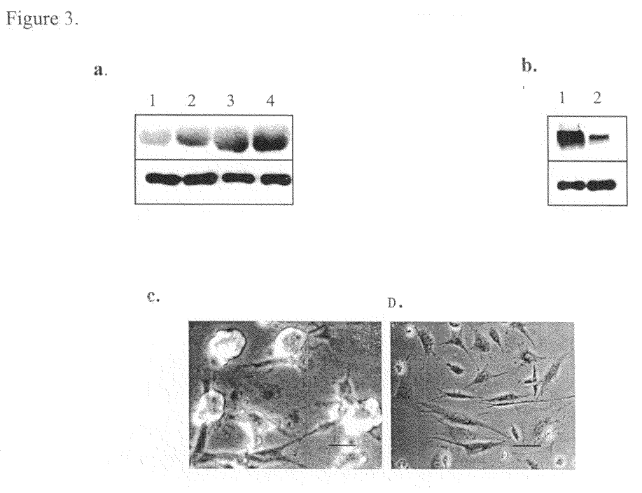

Following transfer of fibroblast cells into SR-containing stem cell medium, stronger Ca²⁺ signalling and up-regulation of the expression of fibroblast growth factor receptor 3 (FGFR3) was observed to occur in a time-dependent manner (FIGS. 3a & 3b). (Up-regulation of FGFR3 had been previously shown to play an important role in the reprogramming of primordial germ cells into stem-like cells (Skottman et al. Stem Cell 24:151-167).)

In view of the observed involvement of the FGF signaling pathway, it was then examined whether Ca²⁺ might be an important secondary messenger responsible for regulating the reprogramming of fibroblast cells induced by SR containing medium activation of the FGF signaling pathway. Thapsigargin, a tight-binding inhibitor for sarco/endoplasmic reticulum Ca²⁺ ATPase, was added to the SR-containing medium at a concentration of 1 μg/ml (Durcova-Hills et al. Stem Cell 24:1441). As shown in FIG. 3c, thapsigargin prevented both the up-regulated expression of FGFR3 and the conversion of skin fibroblast cells into stem cell-like cells.

The methylation status of the Oct4 promoter was also examined before and after contacting fibroblast cells with SR medium, and it was observed that SR-containing medium induced demethylation of the Oct4 promoter.

### Example 4

### Characterization of the Induced Pluripotent Stem (iPS) Cells Induced by SR-Containing Medium

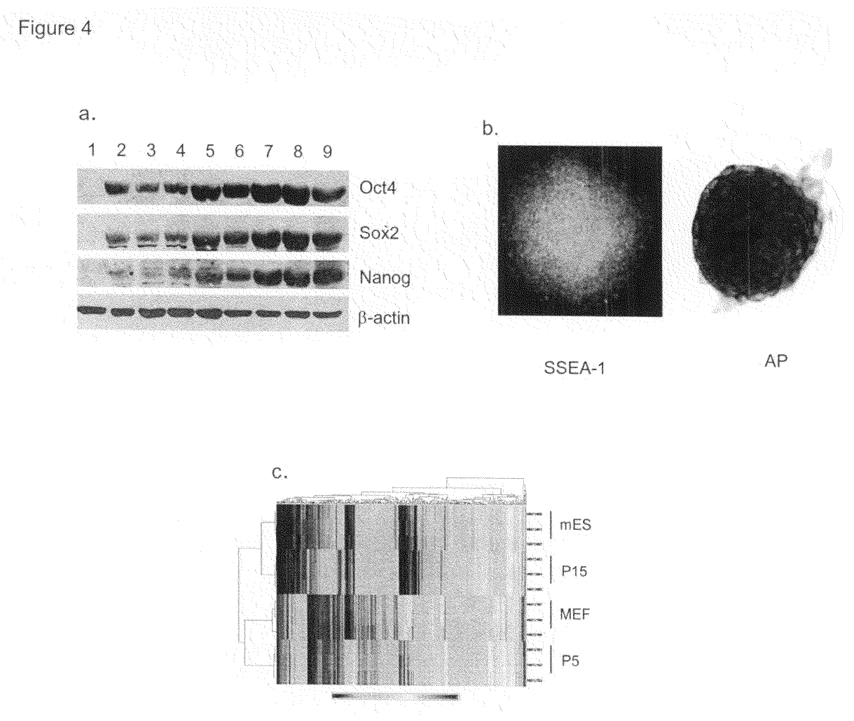

ES cells have been identified to express cell surface markers and factors that distinguish them from differentiated somatic cells. To assess the stem cell qualities of the stem-like cells induced by SR medium treatment of fibroblast cells, the expression levels of the following stem cell-specific markers were measured by Western blot analysis: the POU transcription factor Oct4 (Nichols et al. Cell 95:379-391), the homeodomain protein Nanog (Mitsui et al. Cell 113:631-642), and Sox2 (Avilion et al. Genes Dev. 17:126). As shown in FIG. 4a, the results of this expression analysis demonstrated that serum-replacer-induced pluripotent stem (SR-iPS) cells expressed very high levels of Oct4, Nanog and Sox2 proteins within 2 hours after transfer of the fibroblast cells into SR-containing medium, in contrast to untreated skin fibroblast cells, which did not express detectable levels of Oct4, Nanog and Sox2. To confirm that SR-iPS cells also expressed these typical stem cell factors at the cell surface, SR-iPS cells were fixed and stained with antibodies for alkaline-phosphatase (AP) and stage-specific embryonic antigens 1 (SSEA1 Thomson et al. Science 282:1145-1147). The AP stain result was visible at the early stage of the reprogramming, such as 24 hours after fibroblast transfer into SR-containing medium (FIG. 4b). Only a few cells in the center of the stem cell colony stained red. As passage of the cells continued in SR-medium for 3 or more passages, the whole SR-iPS cell colony showed positive staining with AP and SSEA1, antibodies, as shown in FIG. 4c. These results indicated that the activation of stem cell factors such as Oct4, Nanog, and Sox2 constituted earlier events in the reprogramming process, and that it required additional time to complete reprogramming of the fibroblast cells into stem cell-like cells. Furthermore, global gene expression was examined by microarray analysis in P5 and P115 iES cells, and it was revealed that iES gene expression was very similar to ES cell gene expression, although expression profiles for the two types of cells were not completely identical (FIG. 4d).

### Example 5

### The Pluripotency of SR-iPS Cells

One of the most important characteristics of stem-like cells is pluripotency. To investigate the SR-iPS stem cells have the multilineage differention potential as embryonic stem cells in vitro, different passage of iPS cells were used to form embryo bodis (EBs) one week's culture without passing in SR medium without Lif. The EBs continues to differentiate on gelatin coated plates and induced by 2 μM trans-retinoic acid for an additional 10 days. Expression of endoderm-, mesoderm-, and ectoderm-specific markers was examined by using antibodies raised against α-fetoprotein, smooth muscle actin, and β-tubulin III, respectively (21). For more specific cell lineage such as cardiomyocyte and smooth muscle cells differentiation, EBs were transferred to collagen-coated plate which contained α-MEM (cardiac differentiation medium) supplemented with 10 ng/ml platelet-derived growth factor-BB (PDGF-BB). Vascular endothelial growth medium supplemented with 50 ng/ml vascular endothelial growth factor (VEGF) was used for the smooth muscle growth differentiation. Two weeks after culturing the EBs in these medium, the cell morphology were examined by Immunofluorescent Staining with Troponin C and smooth muscle actin antibodies.

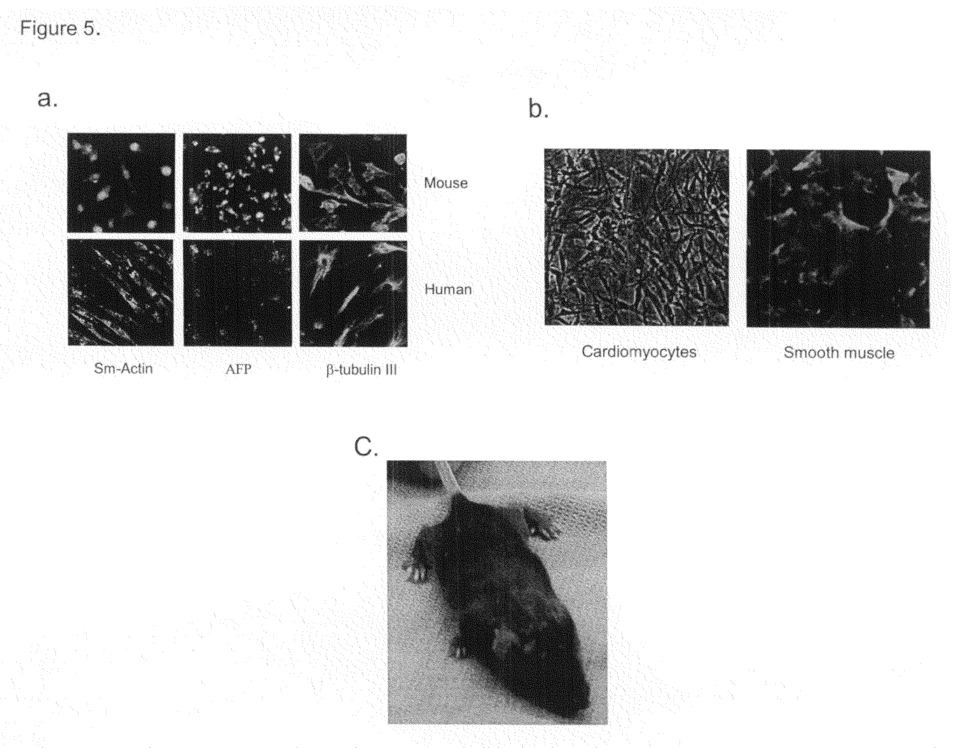

To examine whether SR-iPS stem-like cells possessed developmental potential identical to that of embryonic stem-like cells in vivo, P15 SR-iPS cells were injected into blastocysts (FIG. 5a). These injected blastocysts gave rise to three newborn pups (FIG. 5a). Two of the pups were observed to have agouti-coloured hairs, demonstrating that iPS cells contributed to functional melanocytes. These chimaeric mice appeared healthy and grew normally into adult mice, demonstrating that the SR-containing serum-free medium had reprogrammed treated somatic cells into pluripotent stem-like cells that contributed to blastocysts that produced full term, healthy animals.

### Example 6

### Production and Culturing of Human Fibroblast Stem-Like Cells

To make human stem-like cells, human dermal skin fibroblast were cultured in DMEM medium with 10% FBS at 37 C 5% or 10% CO2 until the cells were confluent.

Serum replacement medium and 0.25% trypsin/EDTA was warmed in 37 C water bath. The media was aspirated off the culture plate and the cells were washed with 1×PBS. 2 ml of trypsin/EDTA was added for 1 minute, and then removed. The trypsin-treated cells were incubated at room temperature for additional 5 minutes, and suspended in 3 ml SR containing medium.

The cells were suspended up and down gently with 5 ml pipet several times until no large clumps remain.

0.5 ml of the cell suspension (about 10⁵ cells/well) was added to each well in a 6 well plate. Each well contained 3 ml SR containing medium.

The cells were placed in an incubator 37 C and 5% or 10% CO2. After the cells were transferred into the SR containing medium, the cells become round, bright edged granulated cells and attached to the bottom of the plate.

Newly converted iPSC (induced Pluripotent stem cell, i.e., stem-like cells) were incubated for 3 to 20 day with the SR containing medium renewed every 2 or 3 days. After the incubation, the ihPSC will continue to be passed every 4 to 6 days depend on the colony size until they become fully pluripotent stage which is determined by stem cell factors expression, gene expression profiles, epigenetic state and differentiation potential.

To pass human stem-like cells, the cells were washed once with 1×PBS. 1 ml of Collogase IV (Invitrogen IV cat# 17104-019.1 mg/ml) was added and incubate 15 minute at 37 C. Resuspend the cells in 1 ml of SR containing medium gently with 5 ml pipet. Centrifuged for 3 minutes at 500 g to remove collogase IV. The cells were resuspended in 1 ml of SR containing medium gently with 5 ml pipet, and then transferred into a new 6 well plate containing 3 ml SR containing medium. The cells are passed at a ratio of between 1:2 and 1:3 according to the colony density.

The ingredients of SR containing medium: 400 ml DMEM/F12 (Invitrogen 11330-032), 5 ml non-Essential Amino Acids (Invitrogen 11140-050), 2.5 ml L-Glutamine (Invitrogen 25030-018), 0.1 μM β-mercaptoethanol (Sigma 7522, add 3.5 ul) and 100 ml Knockout Serum Replacer (Invitrogen 10828-028). 4 ng/mL bFGF (R&D System Cat# 233-FB), 1000/ml unit of ESGRO (Lif) (Chemicon Cat #ESG1107).

### Example 7

### Production and Culturing of Mouse Fibroblast Stem-Like Cells

To make mouse stem-like cells, mouse adult skin fibroblasts or mouse emborynic skin fibroblasts were cultured in DMEM medium with 10% FBS at 37 C 5% or 10% CO2 until the cells were confluent.

Serum replacement medium and 0.25% trypsin/EDTA was warmed in 37 C water bath. The media was aspirated off the culture plate and the cells were washed with 1×PBS. 2 ml of trypsin/EDTA was added for 1 minute, and then removed. The trypsin-treated cells were incubated at room temperature for additional 5 minutes, and suspended in 3 ml SR containing medium.

The cells were suspended up and down gently with 5 ml pipet several times until no large clumps remain.

0.5 ml of the cell suspension (about 10⁵ cells/well) was added to each well in a 6 well plate. Each well contained 3 ml SR containing medium.

The cells were placed in an incubator 37 C and 5% or 10% CO2. After the cells were transferred into the SR containing medium, the cells become round, bright edged granulated cells and attached to the bottom of the plate.

Newly converted iPSC (induced Pluripotent stem cell, i.e., stem-like cells) were incubated for 3 to 20 day with the SR containing medium renewed every 2 or 3 days. After the incubation, the ihPSC will continue to be passed every 4 to 6 days depend on the colony size until they become fully pluripotent stage which is determined by stem cell factors expression as described herein, gene expression profiles, epigenetic state and differentiation potential.

To pass mouse stem-like cells, the cells were washed with 1×PBS, and 1 ml of 0.25% trypsin/EDTA was added to the 6 well culture plate. The trypsin was completely removed after 1 minute. The trypsin-treated cells were incubated at room temperature for additional 5 minutes. The cells are resuspended in 1 ml of SR containing medium gently with 5 ml pipet, and then transferred into a new 6 well plate containing 3 ml SR containing medium. The cells are passaged at a ratio of between 1:2 and 1:3 according to the colony density.

The ingredients of SR containing medium: 400 ml DMEM/F12 (Invitrogen 11330-032), 5 ml non-Essential Amino Acids (Invitrogen 11140-050), 2.5 ml L-Glutamine (Invitrogen 25030-018), 0.1 μM β-mercaptoethanol (Sigma 7522, add 3.5 ul) and 100 ml Knockout Serum Replacer (Invitrogen 10828-028). 4 ng/mL bFGF (R&D System Cat# 233-FB), 1000/ml unit of ESGRO (Lif) (Chemicon Cat #ESG1107).

### Example 8

### Investigation of the Key Components of SR Media

In order to evaluate which components of SR media were important to the production of stem-like cells, an analysis were preformed to determine which components of SR media promoted the formation of stem-like cells.

The results are set forth in Table 1.

TABLE 1
Key components in the “Serum Replacer” promoted the reprogramming
of skin fibroblast cells into stem cell-like cells

| | Ingredient | | | | | | | |
|---|---|---|---|---|---|---|---|---|
| | | Reduced | Ascorbic | | | | | |
| | Thiamine | Glutathione | acid-2 | Transferrin | Insulin | AlbuMAX I | bFGF | Conversion |
| Concentration | 9 | 1.5 | 50 | 8 | 10 | 12,500 | 4 ng mL | |
| in 1N medium | | | | | | | | |
| (mg/L) | | | | | | | | |
| Medium 1 | • | + | + | + | + | + | + | Yes |
| Medium 2 | + | • | + | + | • | + | + | Yes |
| Medium 3 | + | + | • | + | + | + | + | Yes |
| Medium 4 | + | + | + | • | + | + | + | Yes |
| Medium 5 | + | + | + | + | • | + | + | Yes |
| Medium 6 | + | + | + | + | + | • | + | No |
| Medium 7 | + | + | + | + | + | + | • | Yes |

The result in the table showed that the medium 6 without the AlbuMAX I had not the ability to reprogram the somatic cells into stem-like cells. AlbuMAX I is the high-lipid BSA. Several BSA-associated lipids were added to the purified BSA. Our results indicated that acarchodonic acid and high-lipid BSA are required to create stem-like cells

### Example 9

### Expression of Genes in Stem-Like Cells

In order to determine what genes are differentially expressed in the stem-like cells of the invention, Mouse stem-like cells produced by the methods described herein were evaluated. Table II demonstrates that most differentially expressed genes in the stem-like cells.

Using the JA00648 and JA00678 Differentially Expressed Gene (DEG) lists, the top 500 DEG and extracted genes were evaluated where the iPSC expression was VERY different from both MEF and mESC expression. When looking at the two lists, 21 genes appeared on BOTH lists (i.e., the iPSC gene expression was very different from mESC and MEF in both sets in a consistent manner). Eighteen (18) of these genes were UP-regulated in the iPSC, and 3 were DOWN-regulated in the iPSC, as compared to the MEF and mESC.

| | | |
|---|---|---|
| DEFINITION | ACCESSION | SYMBOL |

Mouse iPSC genes UP-regulated

| | | |
|---|---|---|
| Mus musculus calcium/calmodulin-dependent | NM_133926.1 | Camk1 |
| protein kinase I (Camk1), mRNA. | | |
| Mus musculus fatty acid binding protein 3, muscle | NM_010174.1 | Fabp3 |
| and heart (Fabp3), mRNA. | | |
| Mus musculus adenosine deaminase (Ada), | NM_007398.2 | Ada |
| mRNA. | | |
| Mus musculus RIKEN cDNA D630035O19 gene | NM_145932 | D630035O19Rik |
| (D630035O19Rik), mRNA. | | |
| Mus musculus protein kinase C and casein kinase | NM_011861.1 | Pacsin1 |
| substrate in neurons 1 (Pacsin1), mRNA. | | |
| Mus musculus homeo box A5 (Hoxa5), mRNA. | NM_010453.2 | Hoxa5 |
| Mus musculus lipoprotein lipase (Lpl), mRNA. | NM_008509.1 | Lpl |
| Mus musculus RIKEN cDNA D930023J19 gene | XM_133936.5 | D930023J19Rik |
| (D930023J19Rik), mRNA. | | |
| Mus musculus aurora kinase C (Aurkc), mRNA. | NM_020572.1 | Aurkc |
| Mus musculus left-right determination, factor B | NM_010094.2 | Leftb |
| (Leftb), mRNA. | | |
| Mus musculus RIKEN cDNA A130092J06 gene | NM_175511.2 | A130092J06Rik |
| (A130092J06Rik), mRNA. | | |
| Mus musculus SRY-box containing gene 21 | NM_177753.2 | Sox21 |
| (Sox21), mRNA. | | |
| Mus musculus RIKEN cDNA 1700019N12 gene | NM_025953.1 | 1700019N12Rik |
| (1700019N12Rik), mRNA. | | |
| Mus musculus RIKEN cDNA 1700007K13 gene | XM_130125.3 | 1700007K13Rik |
| (1700007K13Rik), mRNA. | | |
| Mus musculus CTD (carboxy-terminal domain, | NM_026295.2 | Ctdp1 |
| RNA polymerase II, polypeptide A) phosphatase, | | |
| subunit 1 (Ctdp1), mRNA. | | |
| Mus musculus RIKEN cDNA 1600023A02 gene | NM_026323.1 | 1600023A02Rik |
| (1600023A02Rik), mRNA. | | |
| Mus musculus aquaporin 3 (Aqp3), mRNA. | NM_016689.1 | Aqp3 |
| | NM_207238.1 | Fbxo27 |

Mouse iPSC genes DOWN-regulated

| | | |
|---|---|---|
| Mus musculus myosin, light polypeptide 4, alkali; | NM_010858.3 | Myl4 |
| atrial, embryonic (Myl4), mRNA. | | |
| Mus musculus early growth response 4 (Egr4), | NM_020596.1 | Egr4 |
| mRNA. | | |
| Mus musculus connective tissue growth factor | NM_010217 | Ctgf |
| (Ctgf), mRNA. | | |

The Illumina Microarray global gene expression analysis of the different passages of SR-iPS cells P5 and P15 compared with mouse embryonic stem cells and MEF cells, the amount 144 genes statistically significant different express (P-ANOVA<0.01154), 129 genes were expressed in a similar fashion between mESC and iPS P15; in the iPS P5 there was less of a similarity to a mES gene expression, with 92 of 144 gene showing similar regulation to MES (17). The results indicated the reprogramming is a gradual process that takes some time to completely inactivate the developmental genes and reactivate the cascade of embryonic stem cell genes. The heat map of top 10,000 differentially expressed genes indicated that at passage 15, the SR-iPS is more similar to embryonic stem cells ES cells, but not completely identical

### INCORPORATION BY REFERENCE

The contents of all references, patents, pending patent applications and published patents, cited throughout this application are hereby expressly incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed is:

1. A method of producing a stem-like cell comprising:
culturing a somatic cell in the presence of arachadonic acid and serum albumin (SA);
thereby producing a stem-like cell.

2. The method of claim 1, wherein the stem-like cell is a pluripotent stem-like cell.

3. The method of claim 1, wherein the serum albumin is bovine serum albumin (BSA).

4. The method of claim 3, wherein the BSA is high lipid BSA.

5. The method of claim 1, wherein the arachodonic acid BSA are present in a serum replacement (SR) medium.

6. The method of claim 1, wherein the somatic cell is a fibroblast.

7. The method of claim 1, wherein the fibroblast is a human fibroblast.

8. The method of claim 7, wherein the fibroblast is a human dermal skin fibroblast.

9. The method of claim 1, wherein the fibroblast is a mouse fibroblast.

10. The method of claim 9, wherein the fibroblast is a mouse embryonic skin fibroblast.

11. The method of claim 9, wherein the fibroblast is a mouse adult skin fibroblast.

12. The method of claim 1, further comprising contacting the cultured cells with a protease.

13. The method of claim 12, wherein the protease is a trypsin.

14. A method of producing a pluripotent stem-like cell comprising:
culturing a somatic cell in the presence of arachadonic acid and serum albumin (SA);
thereby producing a pluripotent stem-like cell.

15. The method of claim 14, wherein the serum albumin is bovine serum albumin (BSA).

16. The method of claim 14, wherein the BSA is high lipid BSA.

17. The method of claim 14, wherein the arachodonic acid BSA are present in a serum replacement (SR) medium.

18. The method of claim 14, wherein the somatic cell is a fibroblast.

19. The method of claim 18, wherein the fibroblast is a human fibroblast.

20. The method of claim 19, wherein the fibroblast is a human dermal skin fibroblast.

21. The method of claim 14, wherein the fibroblast is a mouse fibroblast.

22. The method of claim 21, wherein the fibroblast is a mouse embryonic skin fibroblast.

23. The method of claim 21, wherein the fibroblast is a mouse adult skin fibroblast.

24. The method of claim 14, further culturing the somatic cell in the presence of bFGF.

25. A stem-like cell produced by the method of claim 1 or 14.

26. A pluripotent stem-like cell produced by the method of claim 14.

27. A pluripotent stem-like cell derived from a somatic cell.

28. The stem cell of claim 27 wherein the somatic cell is a fibroblast.

29. A method of treating a subject comprising:
contacting the cell of claim 25 with a tissue-specific growth factor; and administering the cells contacted with the growth factor to the subject.

30. A method of treating a subject comprising:
administering to a subject a stem cell derived by the method of claims 1 or 14,
thereby treating the subject.

31. The method of claim 30, wherein the method further comprises contacting the stem cell with an agent that induces differentiation of the cell into a desired cell type.

32. The method of claim 31, wherein the subject has cancer.

33. A kit comprising an agent to dedifferentiate a somatic cell into a pluripotent stem-like cell and instructions for use.
